(19) United States
(12) Patent Application Publication
(10) Pub. No.: US 2010/0152433 A1
(43) Pub. Date: Jun. 17, 2010

(54) ANTIVIRAL AGENT

(75) Inventors: Michinori Kohara, Tokyo (JP); Shin-ichiro Nakagawa, Tsukuba-shi (JP)

Correspondence Address: DARBY & DARBY P.C., P.O. BOX 770, Church Street Station, New York, NY, 10008-0770, US

(73) Assignee: Michinori Kohara, Tokyo (JP)

(21) Appl. No.: 12/531,735

(22) PCT Filed: Mar. 18, 2008
(86) PCT No.: PCT/JP2008/054946

§ 371 Date: Feb. 25, 2010

(30) Foreign Application Priority Data
Mar. 19, 2007 (JP) 2007-070028
Jan. 28, 2008 (JP) 2008-015713

(51) Int. Cl. C07H 19/20

(52) U.S. Cl. 536/26.3

(57) ABSTRACT

The main purpose of the present invention is to provide a novel antiviral agent having a useful pharmacological action. The present inventors found that the above-described purpose can be achieved by a complex in which two synthetic RNAs (e.g., poly-I and poly-C) that can together form a double strand are contained in a drug carrier useful for transporting a drug into a cell (e.g., cationic liposome and atelocollagen), and thus the present invention was achieved.

### TECHNICAL FIELD

The present invention relates to an antiviral agent.

In this regard, “I”, “C”, “A” and “U” mean inosinic acid, cytidylic acid, adenylic acid and uridylic acid, respectively. Further, as known well in the art, a poly-I analog, poly-C analog, poly-A analog and poly-U analog refer to products in which all or a part of a sugar, nucleobase and phosphate backbone, which constitute a nucleic acid, are modified for the purpose of, for example, enhancing an effect and improving the stability.

### BACKGROUND ART

It is known that, when administered to a living body, a synthetic RNA, which is typified by poly-I, poly-C, poly-A and poly-U, induces type I interferon (hereinafter referred to as “type I IFN”), and that viral growth is suppressed by type I IFN. However, in general, the action of a synthetic RNA to suppress viral growth is insufficient. Therefore, it is thought to be difficult to develop a synthetic RNA as an antiviral agent. In addition, there is concern for toxicity of a synthetic RNA.

It has been proposed that hepatitis should be treated not by using a synthetic RNA alone, but by using a complex formed by a synthetic RNA and a so-called cationic liposome (for example, see Patent Document 1). Such a complex specifically accumulates in the liver of a mouse and induces type I IFN therein, and IFN in the blood reaches a level at which the long-term clinical effectiveness can be expected sufficiently. Therefore, effectiveness of therapy of viral hepatitis was expected. However, the publication only discloses the action mediated by type I IFN induction caused by the complex. At that time, there was a limitation on utilization of a model of viral hepatitis, and the anti-hepatitis virus activity of such a complex had not been confirmed. For example, hepatitis C virus (hereinafter referred to as “HCV”), which is one of hepatitis viruses, only infects liver cells of human and chimpanzee. For this reason, it was virtually impossible to prove how much degree of anti-HCV activity such a complex has using an animal model infected with HCV. However, recently, German and Canadian groups have developed a chimeric mouse having human normal liver cells in its liver. This chimeric mouse with human liver cells has a property of being infected with HCV. Therefore, it enables utilization as a practical animal assessment system for developing an anti-HCV agent. Moreover, since this chimeric mouse can also be infected with hepatitis B virus (hereinafter referred to as “HBV”), it can also be utilized as an animal assessment system for developing an anti-HBV agent.

Patent Document 1: International Publication WO 99/48531 pamphlet

### DISCLOSURE OF THE INVENTION

The present inventors made a comparison between the anti-HCV activity of the above-described complex and the anti-HCV activity of polyethylene glycol (PEG)-attached interferon (hereinafter referred to as “PEGylated IFN”), which is currently most often used as an anti-HCV agent, using the above-described chimeric mouse with human liver cells infected with HCV. As a result, the complex had a stronger anti-HCV activity compared to PEGylated IFN. Further, even when the complex was administered, unlike the case of mouse liver, almost no IFN-β was induced in human liver cells. This indicates that the complex induces a new antiviral mechanism independent of induction of type I IFN.

In addition, the present inventors made a comparison between the anti-HBV activity of the complex and the anti-HBV activity of a nucleoside-based reverse transcriptase inhibitor, Entecavir (hereinafter referred to as “ETV”), which is currently regarded as the anti-HBV agent exhibiting the highest therapeutic effect, or PEGylated IFN, using the above-described chimeric mouse with human liver cells infected with HBV, and obtained knowledge that the complex has a stronger anti-HBV activity compared to ETV and PEGylated IFN.

The main purpose of the present invention is to provide a novel antiviral agent having a useful pharmacological action.

The present inventors found that the above-described purpose can be achieved by a complex in which two synthetic RNAs (e.g., poly-I and poly-C) that can together form a double strand are contained in a drug carrier useful for transporting a drug into a cell (e.g., cationic liposome and atelocollagen) (hereinafter just referred to as “drug carrier”), and thus the present invention was achieved.

Examples of the present invention include an antiviral agent comprising: a complex in which a poly-I or poly-I analog and a poly-C or poly-C analog are contained in a drug carrier; or a complex in which a poly-A or poly-A analog and a poly-U or poly-U analog are contained in a drug carrier (hereinafter collectively referred to as “the present complex”) (hereinafter referred to as the “antiviral agent of the present invention”).

Hereinafter, the present invention will be described in detail.

The “drug carrier” of the present invention is not particularly limited as long as it is pharmaceutically acceptable, can contain a synthetic RNA, and can transport the contained synthetic RNA into a cell. Examples of such drug carriers include a cationic liposome, atelocollagen and a nanoparticle.

Specifically, examples of the cationic liposome include Oligofectamine (registered trademark), Lipofectin (registered trademark), Lipofectamine (registered trademark), Lipofectamine 2000 (registered trademark), Lipofectace (registered trademark), DMRIE-C (registered trademark), GeneSilencer (registered trademark), TransMessenger (registered trademark), TransIT TKO (registered trademark), and a drug carrier disclosed in International Publication WO 94/19314 pamphlet, i.e., a drug carrier formed to comprise a compound represented by the general formula [1] below [e.g., 2-O-(2-diethylaminoethyl)carbamoyl-1,3-O-dioleoylglycerol (hereinafter referred to as “Compound X”), 3-O-(4-dimethylaminobutanoyl)-1,2-O-dioleylglycerol, 3-O-(2-dimethylaminoethyl)carbamoyl-1,2-O-dioleylglycerol, and 3-O-(2-diethylaminoethyl)carbamoyl-1,2-O-dioleylglycerol] and a phospholipid as essential constituents (hereinafter referred to as “the present glycerol carrier”),

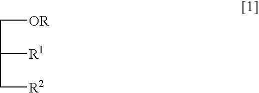

wherein in the formula, R¹ and R² differently represent OY or -A-(CH₂)n-E,

wherein n is an integer from 0 to 4,

E represents pyrrolidino, piperidino, substituted or unsubstituted piperazino, morpholino, substituted or unsubstituted guanidino, or

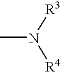

wherein R³ and R⁴ identically or differently represent hydrogen, lower alkyl having 1 to 4 carbon atoms, hydroxy lower alkyl having 1 to 4 carbon atoms, or mono- or di-lower alkylamino alkyl (having 2 to 8 carbon atoms),

A represents the following formula (1), (2), (3), (4), (5), (6) or (7):

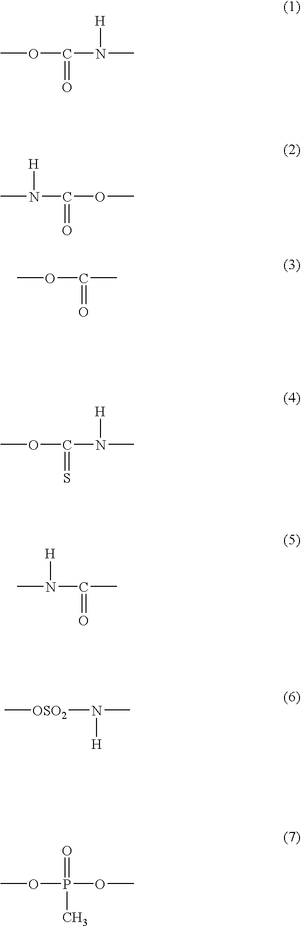

and R and Y identically or differently represent a saturated or unsaturated aliphatic hydrocarbon group having 10 to 30 carbon atoms or a saturated or unsaturated fatty acid residue having 10 to 30 carbon atoms.

In the present invention, examples of preferred cationic liposomes include a drug carrier formed to comprise Compound X and a phospholipid as essential constituents (hereinafter referred to as “the present glycerol carrier X”).

The poly-I analog, poly-C analog, poly-A analog and poly-U analog are not particularly limited as long as the function of the original nucleic acid (for example, poly-I in the case of poly-I analog) is not impaired. Specific examples thereof include poly(7-deazainosinic acid), poly(2′-azidoinosinic acid), poly(cytidine-5′-thiophosphoric acid), poly(1-vinylcytidylic acid), poly(cytidylic acid, uridylic acid)copolymer, poly(cytidylic acid, 4-thiouridylic acid)copolymer, and poly(adenylic acid, uridylic acid)copolymer.

The chain lengths of poly-I, poly-I analog, poly-C, poly-C analog, poly-A, poly-A analog, poly-U and poly-U analog are not particularly limited, but it is suitable that the chain lengths are each independently within the range of 50 to 2,000 bases, preferably within the range of 100 to 600 bases, and more preferably within the range of 200 to 500 bases. The effect of the present invention can be exerted even if the chain lengths are less than 50 bases or more than 2,000 bases. However, when the chain lengths are less than 50 bases, there is a possibility that the problem of effectiveness may arise, and when the chain lengths are more than 2,000 bases, there is a possibility that it may cause toxicity.

Synthetic RNAs such as poly-I and poly-C are usually within a certain distribution consisting of various chain lengths. Accordingly, each of the aforementioned chain lengths means the number of bases with the largest distribution.

The phospholipid in the present glycerol carrier is not particularly limited as long as it is a pharmaceutically acceptable phospholipid. Specific examples thereof include phosphatidylcholine, phosphatidylethanolamine, phosphatidylinositol, phosphatidylserine, sphingomyelin and lecithin. In addition, a hydrogenated phospholipid is also included therein. Preferred examples of the phospholipid include egg-yolk phosphatidylcholine, egg-yolk lecithin, soybean lecithin and egg-yolk phosphatide. One or more of these phospholipids can be used. Regarding the present glycerol carrier X, the same phospholipids as above may be exemplified. Preferred examples of the phospholipid in the present glycerol carrier X also include egg-yolk phosphatidylcholine, egg-yolk lecithin, soybean lecithin and egg-yolk phosphatide. Similarly, one or more of these phospholipids can be used.

Accordingly, preferred examples of the present invention include the antiviral agent of the present invention comprising: a complex in which a poly-I or poly-I analog and a poly-C or poly-C analog (the chain length of each of these synthetic RNAs is within the range of 100 to 600 bases) are contained in the present glycerol carrier X in which the phospholipid is lecithin; or a complex in which a poly-A or poly-A analog and a poly-U or poly-U analog (the chain length of each of these synthetic RNAs is within the range of 100 to 600 bases) are contained in the present glycerol carrier X in which the phospholipid is lecithin. Particularly preferred examples of the present invention include the antiviral agent of the present invention comprising a complex in which a poly-I and poly-C (the chain length of each of these synthetic RNAs is within the range of 200 to 500 bases) are contained in the present glycerol carrier X in which the phospholipid is lecithin.

The ratio between the drug carrier and the synthetic RNAs such as poly-I and poly-C to constitute the present complex varies depending on the type of the drug carrier to be used, the type and the chain length of the synthetic RNAs, the type and the degree of growth of a virus, etc. However, per 10 parts by weight of the drug carrier, it is suitable that the amount of the synthetic RNAs is 0.05 to 10 parts by weight, preferably 0.1 to 4 parts by weight, and more preferably 0.3 to 2 parts by weight.

The ratio between Compound X and the phospholipid to constitute the present glycerol carrier X varies depending on the type and the chain length of the synthetic RNAs, the amount for use, the type of the phospholipid, etc. However, per 1 part by weight of Compound X, it is suitable that the amount of the phospholipid is 0.1 to 10 parts by weight, preferably 0.5 to 5 parts by weight, and more preferably 1 to 2 parts by weight.

The antiviral agent of the present invention may be in the form of, for example, a liquid agent (e.g., injectable drug and drops) or a lyophilized formulation thereof.

The antiviral agent of the present invention may comprise an appropriate amount of any pharmaceutically acceptable additive such as an auxiliary emulsifying agent, a stabilizing agent, a tonicity agent and a pH adjuster. Specific examples thereof include: fatty acid having 6 to 22 carbon atoms (e.g., caprylic acid, capric acid, lauric acid, myristic acid, palmitic acid, stearic acid, oleic acid, linoleic acid, arachidonic acid and docosahexaenoic acid); pharmaceutically acceptable salts thereof (e.g., sodium salt, potassium salt and calcium salt); auxiliary emulsifying agents such as albumin and dextran; stabilizing agents such as cholesterol and phosphatidic acid; tonicity agents such as sodium chloride, glucose, maltose, lactose, sucrose and trehalose; and pH adjusters such as hydrochloric acid, sulfuric acid, phosphoric acid, acetic acid, sodium hydroxide, potassium hydroxide and triethanolamine.

The antiviral agent of the present invention can be produced by subjecting a drug carrier or raw material compounds thereof and synthetic RNAs to mixing, stirring, dispersing or the like according to the ordinary method. In the case of the antiviral agent of the present invention in which the drug carrier is a cationic liposome, it can be produced using, for example, a method similar to the general method for producing a liposome. Specifically, the above-described antiviral agent of the present invention can be produced by subjecting a cationic liposome or raw material compounds thereof (e.g., Compound X and phospholipid) and, for example, double-stranded poly-I and poly-C or a single-stranded poly-I and single-stranded poly-C to dispersion treatment in an aqueous solution using an appropriate disperser. Examples of the aqueous solution include water for injection, distilled water for injection, electrolyte liquid such as saline, and dextrose solution. Examples of the appropriate disperser include a homomixer, a homogenizer, an ultrasonic dispersion machine, an ultrasonic homogenizer, a high-pressure emulsification and dispersion machine, Microfluidizer (trade name), Nanomizer (trade name), Ultimizer (trade name), DeBEE (trade name), and Manton-Gaulin high-pressure homogenizer. Further, the dispersion treatment can be carried out in a stepwise manner (including, for example, coarse dispersion).

As the drug carrier, a commercially available one can be used according the instructions thereof, or such a product can be suitably processed for use.

When producing the antiviral agent of the present invention from raw material compounds of a cationic liposome, for example, double-stranded poly-I and poly-C or a single-stranded poly-I and single-stranded poly-C are added to the raw material compounds and these materials can be subjected to dispersion treatment at a time. Alternatively, the raw material compounds are subjected to dispersion treatment firstly to form a cationic liposome, and subsequently, for example, double-stranded poly-I and poly-C or a single-stranded poly-I and single-stranded poly-C are added thereto to be subjected to dispersion treatment again, thereby producing the antiviral agent of the present invention.

The above-described any pharmaceutically acceptable additive can be added in a suitable step (before or after dispersion).

The lyophilized formulation of the antiviral agent of the present invention can be produced according to the ordinary method. For example, the antiviral agent of the present invention in the form of a liquid is sterilized, and a predetermined amount thereof is dividedly poured into a vial container. Next, prior freezing is carried out at about −40° C. to −20° C. for about 2 hours. The primary drying is carried out at about 0° C. to 10° C. under reduced pressure, and subsequently the secondary drying is carried out at about 15° C. to 25° C. under reduced pressure to perform lyophilization. After that, in general, the inside of the vial is subjected to substitution with nitrogen gas, and the vial is capped, thereby obtaining the lyophilized formulation of the antiviral agent of the present invention.

In general, the lyophilized formulation of the antiviral agent of the present invention can be redissolved by addition of any appropriate solution (a solution for redissolution) for use. Examples of such solutions for redissolution include water for injection, electrolyte liquid such as saline, dextrose solution, and other general infusion solutions. The amount of the solution for redissolution varies depending on application, etc. and is not particularly limited, but it is suitable that the amount is 0.5 to 2 times greater than the amount of the solution before lyophilization or 500 mL or less.

It is considered that the antiviral agent of the present invention can be used, for example, for hepatitis virus such as type A, type B and type C, RS virus, etc. As is clear from the test examples described later, the antiviral agent of the present invention is stronger than PEGylated IFN, and is effective not only for hepatitis C virus (HCV) of genotype 2 (2a, 2b), but also for that of genotype 1 (1a, 1b). The antiviral agent of the present invention is also effective for HCVs of various genotypes including a hepatitis virus on which IFN does not have much therapeutic effect (IFN-resistant hepatitis virus). In view of the above-described matters, it is expected that a technique like so-called cocktail therapy in HCVs iRNA is not required when using the antiviral agent of the present invention.

The antiviral agent of the present invention is effective for animals including human.

Examples of methods for administering the antiviral agent of the present invention include intravenous administration, subcutaneous administration, hepatic arterial administration, and local administration (e.g., transmucosal administration, transnasal administration and inhalation administration).

The amount of the antiviral agent of the present invention to be administered varies depending on the type and composition of a drug carrier and synthetic RNA to be used, chain length, the type and progression of a virus, the age of a patient, specific difference between animals, administration route, administration method, etc. However, usually, it is suitable that the amount of a synthetic RNA (e.g., poly-I and poly-C) for each administration is 1 μg to 50 mg per human, and preferably 10 μg to 10 mg per human. The antiviral agent of the present invention can be administered by means of one-shot administration, infusion administration or the like once to three times per day everyday, every other day, every week, every other week, or the like.

### BRIEF DESCRIPTION OF THE DRAWINGS

FIG. 1 shows the number of HCV (type 1a) genomes in serum. The numerical values on the horizontal axis show mouse individual numbers, and the numerical values on the vertical axis show numbers of genomes (copies/mL).

FIG. 2 shows the number of HCV (type 1a) genomes in serum. The numerical values on the horizontal axis show mouse individual numbers, and the numerical values on the vertical axis show numbers of genomes (copies/mL).

FIG. 3 shows the number of HCV (type 1b) genomes in serum. The numerical values on the horizontal axis show mouse individual numbers, and the numerical values on the vertical axis show numbers of genomes (copies/mL).

FIG. 4 shows the number of HCV (type 1b) genomes in serum. The numerical values on the horizontal axis show mouse individual numbers, and the numerical values on the vertical axis show numbers of genomes (copies/mL).

FIG. 5 shows the amount of IFN mRNA in liver of chimeric mouse with human liver cells. The numerical values on the horizontal axis show mouse individual numbers, and the numerical values on the vertical axis show amounts of human or mouse IFN-β (copies/μg RNA).

FIG. 6 shows the amount of IFN-β in serum. The numerical values on the horizontal axis show mouse individual numbers, and the numerical values on the vertical axis show concentrations in the blood (pg/mL).

FIG. 7 shows change in the number of HBV genomes in serum. The horizontal axis shows the number of days after start of administration. The vertical axis shows the number of HBV genomes (%) when regarding the number of HBV genomes in serum one day before start of administration (day 1) as 100%.

### BEST MODE FOR CARRYING OUT THE INVENTION

Hereinafter, the present invention will be more specifically described by way of production examples and test examples.

### EXAMPLES

### Production Example 1

124 g of Compound X, 200 g of purified egg-yolk lecithin and 2 kg of maltose were put into a container, and 10.2 L of water for injection was added thereto to be stirred and mixed. After the mixture was subjected to crude emulsification using a homogenizer, it was subjected to fine emulsification using a high-pressure emulsification and dispersion machine (Microfluidizer (registered trademark)). To the obtained mixture, water for injection in which poly-I having about 300 bases and poly-C having about 300 bases were dissolved (8 L in total) was gradually added, and the obtained mixture was dispersed using the high-pressure emulsification and dispersion machine again. The dispersed liquid was filtered with a 0.2 μm membrane filter and sterilized, thereby obtaining the antiviral agent of the present invention. Next, each of vial containers was filled with 5 mL of the antiviral agent of the present invention, and thereafter lyophilized according to the ordinary method, thereby obtaining the lyophilized antiviral agent of the present invention.

### Test Example 1

### Action of Suppression of Viral Growth in HCV-Infected Chimeric Mouse with Human Liver Cells

### (1) Method

As HCV models, chimeric mice with human liver cells infected with genotype 1a HCV according to the ordinary method (manufactured by PhoenixBio Co., Ltd.; the same applies to the following) were used (Takuya Umehara, Masayuki Sudoh, FumihikoYasui, Chiho Matsuda, Yukiko Hayashi, and Michinori Kohara. Serine palmitoyltransferase inhibitor suppresses HCV replication in a mouse model. Biochem. Biophys. Res. Commun. 346, 67-73 (2006)). 4.6 mL of water for injection was added to the lyophilized antiviral agent of the present invention obtained in Production Example 1 for restructuring, and 5% dextrose solution was added thereto to be suitably diluted, thereby preparing a test solution (administration solution).

The test solution was administered to the above-described chimeric mouse by means of tail vein injection so that the dosage amount of poly-I/poly-C became 10 μg/kg, 30 μg/kg or 100 μg/kg. 8-day continuous administration was performed (once per day) from the administration start date (day 0) to 7 days after the start of administration (day 7). As a control drug, 30 μg/kg of PEGylated IFN (Pegasys (registered trademark); manufactured by Chugai Pharmaceutical Co., Ltd.; the same applies to the following) was used in intermittent subcutaneous administration on the administration start day (day 0), 3 days after the start of administration (day 3), 7 days after the start of administration (day 7), and 10 days after the start of administration (day 10). Note that the human clinical dose of PEGylated IFN is 3 μg/kg for once per week.

Total RNA was extracted from the serum which was obtained according to the ordinary method using the acid guanidinium-phenol-chloroform method, and the number of HCV genome copies in the serum was measured using the real time PCR method. Thus, the activity of the antiviral agent of the present invention to suppress viral growth was evaluated.

Regarding the group, in which the antiviral agent of the present invention was administered, the blood was collected 3 days before the start of administration (day −3), one day after the start of administration (day 1), 4 days after the start of administration (day 4), and 8 days after the start of administration (day 8). Regarding the PEGylated IFN-administered group and the control group, the blood was collected one day before the start of administration (day −1), one day after the start of administration (day 1), 8 days after the start of administration (day 8) and 14 days after the start of administration (day 14).

### (2) Results

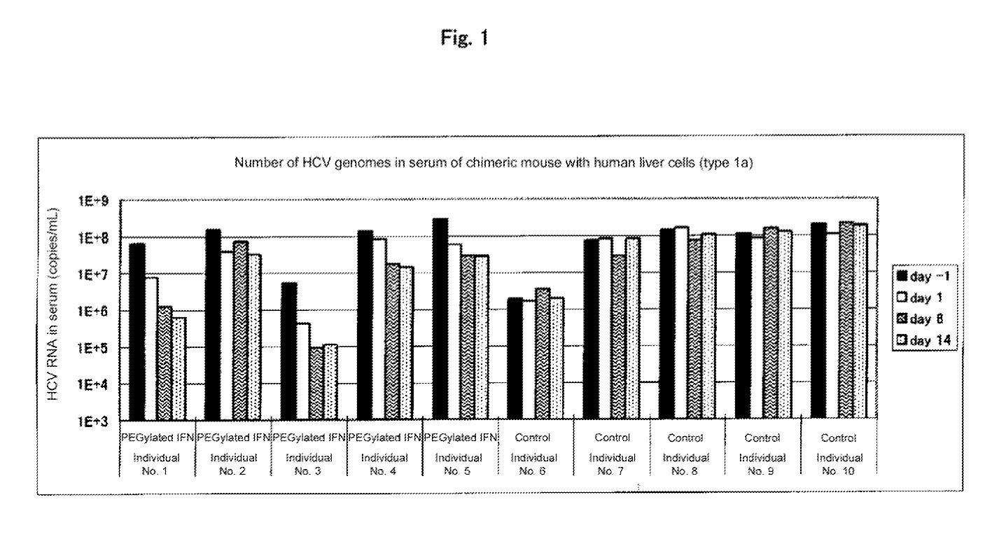

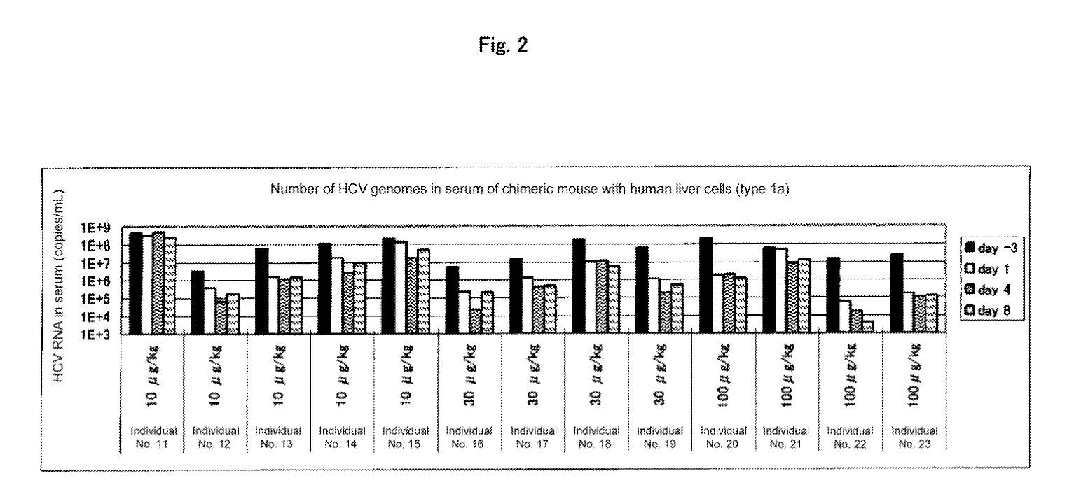

Results are shown in FIGS. 1 and 2.

In the above-described figures: Individual Nos. 1-5 indicate results of the PEGylated IFN-administered group; Individual Nos. 6-10 indicate results of the control (0.9% saline) group; Individual Nos. 11-15 indicate results of the group in which 10 μg/kg of the antiviral agent of the present invention was administered; Individual Nos. 16-19 indicate results of the group in which 30 μg/kg of the antiviral agent of the present invention was administered; and Individual Nos. 20-23 indicate results of the group in which 100 μg/kg of the antiviral agent of the present invention was administered.

As is clear from FIGS. 1 and 2, the antiviral agent of the present invention strongly suppressed the number of HCV genomes in serum in a dose-dependent manner. In the case of the PEGylated IFN-administered group, the amount of HCV was suppressed to 1/10 to 1/100 of the amount before the administration. Meanwhile, in the case of the groups in which the antiviral agent of the present invention was administered; suppression was as follows: 10 μg/k; 1/10 to 1/100; 30 μg/kg; 1/20 to 1/200; and 100 μg/kg; 1/100 to 1/1,000.

In this experiment, there were individuals which did not respond, i.e., Individual No. 2 (belonging to the PEGylated IFN-administered group), Individual No. 11 (belonging to the group in which 10 μg/kg of the antiviral agent of the present invention was administered), and Individual No. 21 (belonging to the group in which 100 μg/kg of the antiviral agent of the present invention was administered).

### Test Example 2

### Action of Suppression of Viral Growth in HCV-Infected Chimeric Mouse with Human Liver Cells

### (1) Method

As HCV models, chimeric mice with human liver cells infected with genotype 1b HCV according to the ordinary method were used (Takuya Umehara, Masayuki Sudoh, Fumihiko Yasui, Chiho Matsuda, Yukiko Hayashi, and Michinori Kohara. Serine palmitoyltransferase inhibitor suppresses HCV replication in a mouse model. Biochem. Biophys. Res. Commun. 346, 67-73 (2006)). 4.6 mL of water for injection was added to the lyophilized antiviral agent of the present invention obtained in Production Example 1 for restructuring, and 5% dextrose solution was added thereto to be suitably diluted, thereby preparing a test solution (administration solution).

The test solution was administered to the above-described chimeric mouse by means of tail vein injection so that the dosage amount of poly-I/poly-C became 100 μg/kg. 8-day continuous administration was performed (once per day or three times per day) from the administration start date (day 0) to 7 days alter the start of administration (day 7). As a control drug, 30 μg/kg of PEGylated IFN was used in intermittent subcutaneous administration on the administration start day (day 0), 3 days after the start of administration (day 3), 7 days after the start of administration (day 7), and 10 days after the start of administration (day 10). Note that the human clinical dose of PEGylated IFN is 3 μg/kg for once per week.

Total RNA was extracted from the serum which was obtained according to the Ordinary method using the acid guanidinium-phenol-chloroform method, and the number of HCV genome copies in the serum was measured using the real time PCR method. Thus, the activity of the antiviral agent of the present invention to suppress viral growth was evaluated.

Regarding the group in which the antiviral agent of the present invention was administered, the blood was collected 2 days before the start of administration (day −2), one day after the start of administration (day 1), 4 days after the start of administration (day 4), and 8 days after the start of administration (day 8). Regarding the PEGylated IFN-administered group and the control group, the blood was collected one day before the start of administration (day −1), one day after the start of administration (day 1), 4 days after the start of administration (day 4), 8 days after the start of administration (day 8), 11 days after the start of administration (day 11), and 14 days after the start of administration (day 14).

### (2) Results

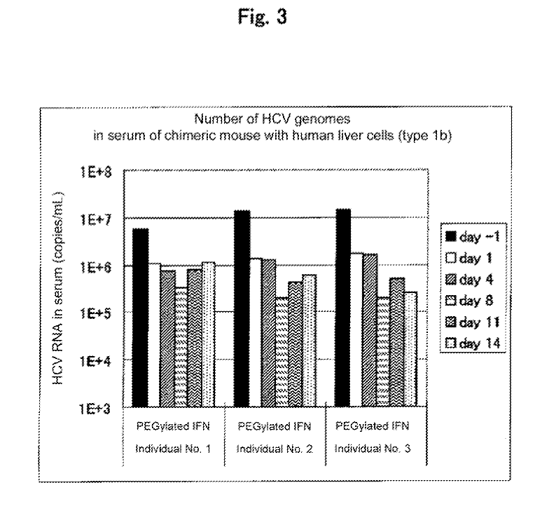

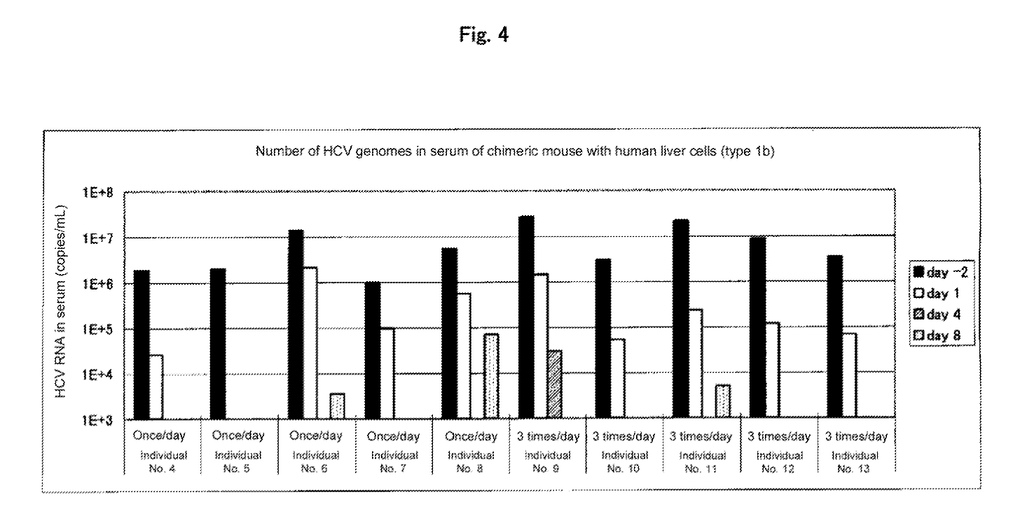

Results are shown in FIGS. 3 and 4.

In the above-described figures: Individual Nos. 1-3 indicate results of the PEGylated IFN-administered group: Individual Nos. 4-8 indicate results of the group in which 100 μg/kg of the antiviral agent of the present invention was administered (once per day); and Individual Nos. 9-13 indicate results of the group in which 100 μg/kg of the antiviral agent of the present invention was administered (three times per day).

As is clear from FIGS. 3 and 4, the antiviral agent of the present invention very strongly suppressed the number of HCV genomes in serum both in the case of the group of administration once per day and the case of the group of administration three times per day. The effect thereof was higher than that exerted by PEGylated IFN, which is currently thought to be the HCV inhibitor having the highest therapeutic effect. In the case of administration of PEGylated IFN, the number of HCV genomes in serum was only suppressed to about 1/100, whereas in the case of the group in which 100 μg/kg (per once) of the antiviral agent of the present invention was administered, the number was suppressed to 1/1,000 to 1/10,000.

In the case of PEGylated IFN, when the administration thereof was stopped, the number of HCV genomes in serum rapidly increased. Meanwhile, in the case of the antiviral agent of the present invention, even 7 days after termination of the administration, the number of individuals was about half, and the effect of strongly suppressing the number of HCV genomes in serum was retained.

### Test Example 3

### Action of Induction of Human and Mouse IFN mRNA in Liver Using the Antiviral Agent of the Present Invention

### (1) Method

4.6 mL of water for injection was added to the lyophilized antiviral agent of the present invention obtained in Production Example 1 for restructuring, and 5% dextrose solution was added thereto to be subjected to 50-fold dilution, thereby preparing a test solution (administration solution, 20 μg/mL). The dose of the test solution for one administration is 100 μL per 20 g body weight of the chimeric mouse with human liver cells (the dose of poly-I/poly-C is 100 μg/kg), and this was intravenously administered through the orbital venous plexus.

Collection of liver and blood was carried out as follows: 2 mouse individuals (Individual Nos. 3 and 4): 2 hours after one administration; another 2 mouse individuals (Individual Nos. 5 and 6): 24 hours after 4-day administration (once per day); and another 3 mouse individuals (Individual Nos. 7-9): 2 hours after 5-day administration (once per day). As the non-treated control group, liver and serum were collected from another 2 mouse individuals (Individual Nos. 1 and 2). At the time of collection of liver, 5 mm-wide sections were cut from 2 lobes of liver.

Total RNA was extracted from the collected liver using the acid guanidinium-phenol-chloroform method, and the number of human/mouse IFN mRNA copies in the liver was measured using the reverse transcription reaction followed by real time PCR. Further, the quantity of human/mouse IFN-β in the serum obtained according to the ordinary method was determined using the ELISA method.

### (2) Results

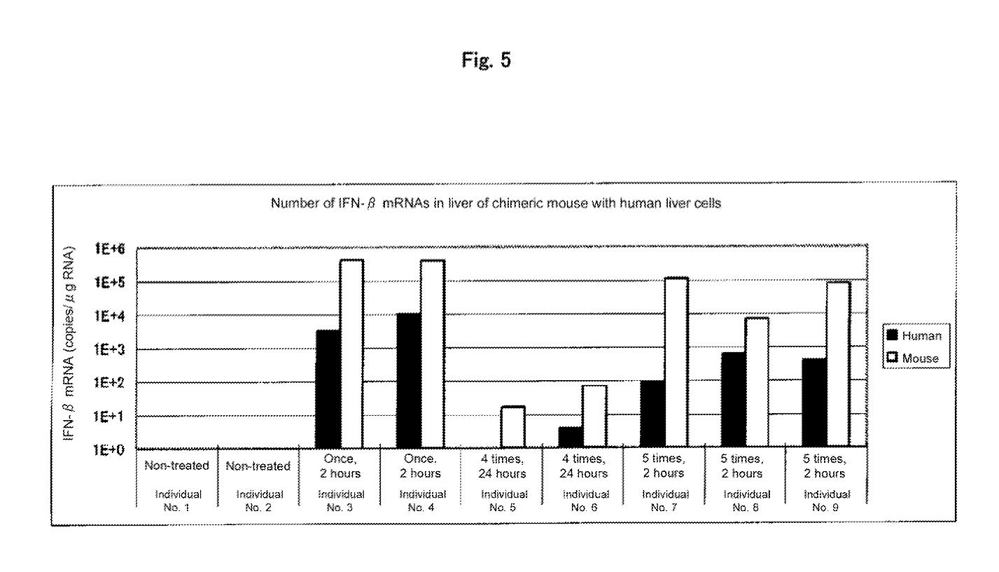

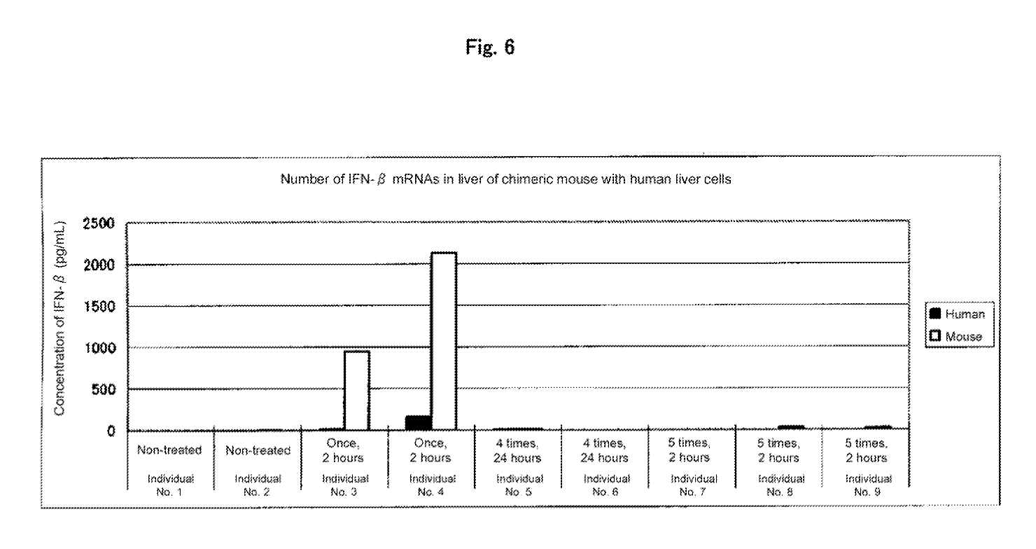

Results are shown in FIGS. 5 and 6.

As is clear from FIGS. 5 and 6, human IFN-β was induced only in an amount that was about 1/100 of that of mouse IFN-β. Thus, human IFN-β was not induced so much, but as indicated by Test Examples 1 and 2, the antiviral agent of the present invention can remove HCV from liver cells.

### Test Example 4

### Action of Suppression of Viral Growth in HBV-Infected Chimeric Mouse with Human Liver Cells

### (1) Method

As HBV models, chimeric mice with human liver cells infected with HBV according to the ordinary method were used (Masaya Sugiyama, Yasuhito Tanaka, Takanobu Kato, Etsuro Orito, Kiyoaki Ito, Subrat K. Acharya, Robert G. Gish, Anna Kramvis, Takashi Shimada, Namiki Izumi, Masahiko Kaito, Yuzo Miyakawa, and Masashi Mizokami. Influence of Hepatitis B Virus Genotypes on the Intra and Extracellular Expression of Viral DNA and Antigens. HEPATOLOGY, 44 (4), 915-924 (2006)).

4.6 mL of water for injection was added to the lyophilized antiviral agent of the present invention obtained in Production Example 1 for restructuring, and 5% dextrose solution was added thereto to be diluted to 20 μg/mL, thereby preparing a test solution (administration solution).

The test solution was administered to the above-described chimeric mouse through the orbital venous plexus so that the dosage amount of poly-I/poly-C became 100 μg/kg. 14-day continuous administration was performed (once per day) from the administration start date (day 0) to 13 days after the start of administration (day 13). As a control drug, 30 μg/kg of PEGylated IFN was used in intermittent subcutaneous administration (once per day) on the administration start day (day 0), 3 days after the start of administration (day 3), 7 days after the start of administration (day 7), and 10 days after the start of administration (day 10). In addition, as another control drug, 17 μg/kg or 170 μg/kg of Entecavir (ETV) (Baraclude (registered trademark); Bristol-Myers Company; the same applies to the following) was used in 14-day continuous oral administration (once per day) from the administration start date (day 0) to 13 days after the start of administration (day 13).

The blood was collected from the orbital venous plexus one day before the start of administration (day −1), one day after the start of administration (day 1), 3 days after the start of administration (day 3), 7 days after the start of administration (day 7), 10 days after the start of administration (day 10), and 14 days after the start of administration (day 14).

DNA was extracted from 1 μL of serum obtained according to the ordinary method using SMITEST (registered trademark) EX-R&D (Medical & Biological Laboratories Co., Ltd.), and the number of HBV genomes in the serum was measured using the real time PCR method, thereby quantifying the number of HBV genomes in the serum.

### (2) Results

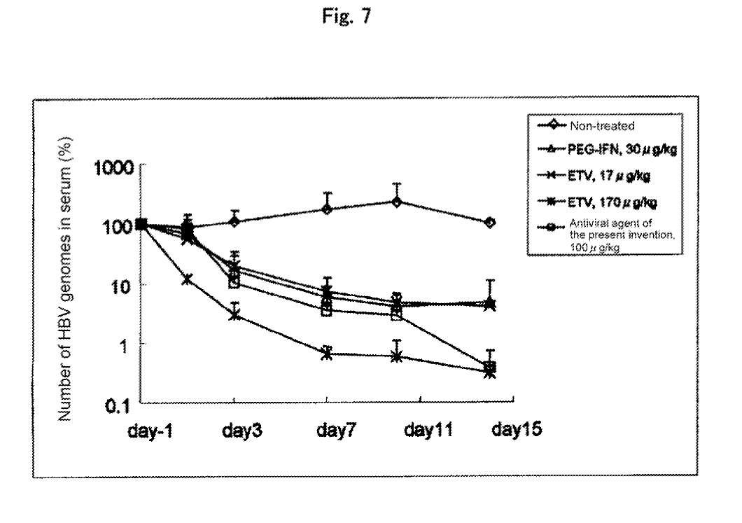

Results are shown in FIG. 7.

As is clear from FIG. 7, when administering PEG-IFN in an amount which is 20 times the clinical dose (30 μg/kg, twice per week), the number of HBV genomes in the serum was suppressed to 1/23 of the number before the start of administration 14 days after the start of administration (day 14). When administering ETV in an amount which is equal to the clinical dose (17 μg/kg, daily administration) or 10 times the clinical dose (170 μg/kg, daily administration), the number was suppressed to 1/25 or 1/320 of the number before the start of administration 14 days after the start of administration (day 14). Meanwhile, in the case of the antiviral agent of the present invention (100 μg/kg, daily administration), the number of HBV genomes in the serum was suppressed to 1/270 of the number before the start of administration 14 days after the start of administration (day 14).

The antiviral agent of the present invention suppressed the number of HBV genomes in the serum more strongly compared to PEGylated IFN in an amount which is 20 times the clinical dose and the clinical dose of ETV. The anti-HBV activity of the antiviral agent of the present invention was equivalent to that of ETV in an amount which is 10 times the clinical dose.

1. An antiviral agent comprising: a complex in which a poly-I or poly-I analog and a poly-C or poly-C analog are contained in a drug carrier useful for transporting a drug into a cell; or a complex in which a poly-A or poly-A analog and a poly-U or poly-U analog are contained in a drug carrier useful for transporting a drug into a cell.

2. The antiviral agent according to claim 1, wherein the drug carrier useful for transporting a drug into a cell is selected from the group consisting of a cationic liposome, atelocollagen and a nanoparticle.

3. The antiviral agent according to claim 2, wherein the cationic liposome is formed to comprise a compound represented by the following general formula [I] and a phospholipid as essential constituents: 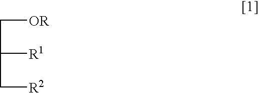
wherein in the formula, R¹ and R² differently represent OY or -A-(CH₂)n-E,
wherein n is an integer from 0 to 4,
E represents pyrrolidino, piperidino, substituted or unsubstituted piperazino, morpholino, substituted or unsubstituted guanidino, or 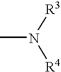
wherein R³ and R⁴ identically or differently represent hydrogen, lower alkyl having 1 to 4 carbon atoms, hydroxy lower alkyl having 1 to 4 carbon atoms, or mono- or di-lower alkylamino alkyl (having 2 to 8 carbon atoms),
A represents the following formula (1), (2), (3), (4), (5), (6) or (7): 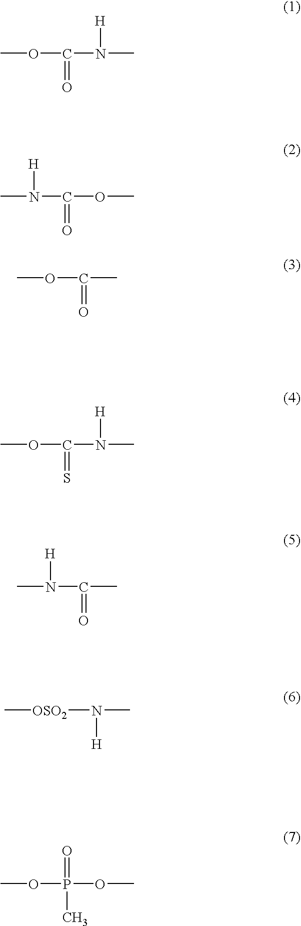
and R and Y identically or differently represent a saturated or unsaturated aliphatic hydrocarbon group having 10 to 30 carbon atoms or a saturated or unsaturated fatty acid residue having 10 to 30 carbon atoms.

4. The antiviral agent according to claim 3, wherein the compound represented by general formula [I] is 2-O-(2-diethylaminoethyl)carbamoyl-1,3-O-dioleoylglycerol, dimethylaminobutanoyl)-1,2-O-dioleylglycerol, 3-O-(2-dimethylaminoethyl)carbamoyl-1,2-O-dioleylglycerol, or 3-O-(2-diethylaminoethyl)carbamoyl-1,2-O-dioleylglycerol.

5. An antiviral agent comprising: a complex in which a poly-I or poly-I analog and a poly-C or poly-C analog are contained in a cationic liposome formed to comprise 2-O-(2-diethylaminoethyl)carbamoyl-1,3-O-dioleoylglycerol and a phospholipid as essential constituents; or a complex in which a poly-A or poly-A analog and a poly-U or poly-U analog are contained in a cationic liposome formed to comprise 2-O-(2-diethylaminoethyl)carbamoyl-1,3-O-dioleoylglycerol and a phospholipid as essential constituents.

6. The antiviral agent according to claim 1, wherein the chain lengths of poly-I, poly-I analog, poly-C, poly-C analog, poly-A, poly-A analog, poly-U and poly-U analog are each independently within the range of 100 to 600 bases.

7. An antiviral agent comprising a complex in which a poly-I having the chain length of 100 to 600 bases and a poly-C having the chain length of 100 to 600 bases are contained in a cationic liposome formed to comprise 2-O-(2-diethylaminoethyl)carbamoyl-1,3-O-dioleoylglycerol and a phospholipid as essential constituents.

8. The antiviral agent according to claim 3, wherein the phospholipid is lecithin.

9. The antiviral agent according to claim 1, wherein the virus is a hepatitis virus.

10. The antiviral agent according to claim 9, wherein the hepatitis virus is a hepatitis C virus.

11. The antiviral agent according to claim 10, wherein the genotype of the hepatitis C virus is type 1a or type 1b.

12. The antiviral agent according to claim 9, wherein the hepatitis virus is a hepatitis B virus.

13. The antiviral agent according to claim 12, wherein the genotype of the hepatitis B virus is type C.

14. The antiviral agent according to claim 5, wherein the chain lengths of poly-I, poly-I analog, poly-C, poly-C analog, poly-A, poly-A analog, poly-U and poly-U analog are each independently within the range of 100 to 600 bases.

15. The antiviral agent according to claim 5, wherein the phospholipid is lecithin.

16. The antiviral agent according to claim 7, wherein the phospholipid is lecithin.

17. The antiviral agent according to claim 5, wherein the virus is a hepatitis virus.

18. The antiviral agent according to claim 7, wherein the virus is a hepatitis virus.
